(19) United States
(12) Patent Application Publication
(10) Pub. No.: US 2004/0092451 A1
(43) Pub. Date: May 13, 2004

(54) Precipitation of growth-factor-enriched fibrinogen concentrate from platelet rich plasma

(76) Inventor: Lou Blasetti, North Quincy, MA (US)

Correspondence Address: CLARK & BRODY, 1750 K STREET NW, SUITE 600, WASHINGTON, DC, 20006, US

(21) Appl. No.: 10/698,404

(22) Filed: Nov. 3, 2003

Related U.S. Application Data
(63) Continuation of application No. 09/509,545, filed on Apr. 28, 2000, now abandoned.
Application No. 09/509,545 is a 371 of international application No. PCT/US98/21626, filed on Oct. 16, 1998.
(60) Provisional application No. 60/062,264, filed on Oct. 17, 1997.

(51) Int. Cl. A61K 38/18; C07K 14/475

(52) U.S. Cl. 514/12; 530/399

(57) ABSTRACT

Increased fibrinogen yields are obtained by adding a precipitating agent to plasma having a high platelet concentration, such as platelet rich plasma. The precipitating agent may be any of several known agents, including polyethylene glycol and ammonium sulfate. The platelet rich plasma is obtained in the preferred embodiment by subjecting plasma to “soft spin” centrifugation of about 580G. An automatic, multiple decanting and multiple-speed centrifuge is preferably used to separate anti-coagulated whole blood into the platelet rich plasma component and red blood cells. The proteins. preferably fibrinogen, FXIII, and FVIII, in the platelet rich component are precipitated, and the proteins and platelets are then concentrated by further centrifugation.

### TECHNICAL FIELD

This application relates to improved processes for recovery and concentration of blood components. In particular. the invention relates to the production of growth-factor-enriched fibrinogen concentrate from platelet-rich plasma.

### BACKGROUND

The need exists for means quickly to concentrate and recover certain blood proteins from whole blood, which also contains platelets and certain growth factors, in a closed-process system for use by physicians to assist in closing wounds, to achieve faster haemostasis, to seal air and fluid leakage, and to aid in faster healing and for drug and biologic delivery.

Those skilled in the art know that when platelet-rich plasma is harvested from a surgical patient intraoperatively and is combined with thrombin, usually in a seven-to-one ratio, and deposited on a wound site, a platelet gel is formed within seconds of application. The gel achieves faster haemostasis than do other conventional haemostatic agents. The gel also seals air and fluid leakage due to its viscous properties, and results in faster healing resulting from the presence of platelet derived growth factors (PDGF). Such a gel contains only native levels of fibrinogen, FXIII, FVIII, and PDGF. Thus, the adhesive, tensil and shear strength of the clot formed by this gel is generally less than is desirable. Further, failure of haemostasis or sealing can occur because of these low levels of desirable proteins, resulting in a failure to achieve the desired outcome.

Harvesting platelet rich plasma from a patient in the intra-operative setting requires a “blood processor,” one of which is sold under the trademark “Cell Saver,” but other devices manufactured by various companies are known. The Cell Saver device requires a highly-skilled. sometimes certified, operator to set-up and operate the device. Operation (which can take 30 to 60 minutes) requires large-bore venous or arterial access and processing of up to several liters of blood to obtain and sequester sufficient platelets and plasma volume. The patient's haemodynamic and cardiac status must be stable to allow processing of such large volumes.

An automated system for obtaining autologous fibrinogen has been described in U.S. Pat. No. 5,707,331 (Wells et al.), the disclosure of which is incorporated herein by reference. According to that system, a relatively small volume (e.g., 50 ml) of whole blood is placed in a first chamber of a two-chamber disposable container. A fibrinogen-precipitating agent is placed in the second chamber. The container is then placed in a centrifuge, and the whole blood is centrifuged to separate the plasma to produce platelet-poor plasma. The platelet-poor plasma, thus obtained is then decanted into the second chamber where it is mixed with the precipitating agent (e.g., PEG or ammonium sulfate). The plasma and precipitating agent are then centrifuged to obtain a pellet of fibrinogen for combination with thrombin to make a fibrin sealant.

### DESCRIPTION OF THE PREFERRED EMBODIMENT.

An important factor for processes that recover fibrinogen, such as the one described in the mentioned U.S. Pat. No. 5,707,331, is the percentage of the fibrinogen in the whole blood that is recovered in the pellet. Applicant has discovered that this factor, the “fibrinogen yield,” is unexpectedly greater when the plasma from which fibrinogen is precipitated contains increased levels of platelets. Thus, according to the process of the invention, a known fibrinogen precipitating agent is added to platelet-rich plasma to obtain increased yields of fibrinogen.

The fibrinogen yield obtained with prior art methods is generally about 50%. whereas the fibrinogen yield obtained in accordance with the methods of the invention is about 72%, which represents a 44% increase in recovered fibrinogen.

In the preferred embodiments, the platelet-rich plasma from which fibrinogen is precipitated contains at least 50K platelets per mm³ and preferably about 200K/mm³.

The disclosed invention produces FVIII and concentrated (up to 10+ fold increase) proteins, preferably fibrinogen, FXIII, and recovered platelets (and resultant increase in human growth factors) from relatively small aliquots (20 cc-150 cc) of anti-coagulated whole blood in a short time (approx. 20 minutes). The increased coagulation protein concentration of the disclosed invention over the current Cell Saver methods results in a clinically more effective (greater tensile and shear strength) clot. A clinically effective dose is produced from a smaller volume (20 cc-150 cc) of the patient's blood obtained by simple phlebotomy known in the art versus the Cell Saver method (several liters):

The preferred method utilizes the dedicated centrifuge and disposable container described in U.S. Pat. No. 5,707,331 to process anti-coagulated whole blood drawn from a patient (or directed blood donor). In accordance with the invention, the process described there is modified to provide platelet-rich plasma by appropriate control of the centrifuge speed and the length of time the blood is subjected to centrifugation.

Anticoagulated blood retrieved from a mammal by simple phlebotomy techniques is dispensed into a first chamber of a 2-chamber disposable, and an appropriate volume of a precipitating agent, for example PEG or saturated ammonium sulfate, is placed in the second chamber. The ammonium sulfate can be 25% to 100% ammonium sulfate, and is preferably about 95% ammonium sulfate. The disposable is loaded into the dedicated centrifuge as described in U.S. Pat. No. 5,707,331, and the process in that patent initiated. The centrifuge is programmed to effect the following steps automatically:

1. Red cells are separated from whole blood in the centrifuge at a spin rate that produces platelet-rich plasma (PRP). The spin rate is known as a “soft spin” and preferably one that produces about 580G. The centrifuge is then stopped, and the PRP is decanted from the first chamber to the second, where it is mixed with the precipitating agent. This soft spin has been found to produce plasma having a platelet concentration of from about 50K/mm³ to about 450K/mm³.

2. After mixing is complete, the centrifuge re-starts and the precipitated proteins, along with the platelets, are concentrated by a “hard spin,” preferably one that produces about 3500G.

3. Following step 2 above, the platelet-poor and fibrinogen-poor plasma and residual precipitating agent are decanted from the second chamber back to the first. leaving a relatively-dry, growth-factor-enriched protein/platelet pellet. The use of a precipitating agent, such as PEG or ammonium sulfate, with PRP has been found to provide greater protein (preferably fibrinogen) recovery than obtained with techniques using a precipitating agent with platelet poor plasma (PPP).

4. A suitable diluent volume, preferably a citrate buffer, is added to re-dissolve and recover the protein/platelet pellet to allow transport by, for example, syringe.

5. When the recovered, concentrated protein, containing increased levels of human growth factors, is combined with thrombin and deposited on a wound site, a platelet gel is formed within seconds of application. The gel achieves faster haemostasis than when other conventional haemostatic agents are used. It can also seal air and fluid leakage due to its viscous properties, and results in faster healing from the presence of enriched platelet derived growth factors (PDGF). The gel's properties include FVIII and increased levels of fibrinogen, FXIII, and greater than native levels of human growth factors. These increased levels result in a clot with more desirable adhesive, tensile and shear strength. Because of these higher levels of desirable proteins, the risk of premature failure of the clot is reduced and the likelihood of achieving the desired outcome is increased.

### EXAMPLE 1

Fifty milliliters of whole blood were placed in the first chamber of a container for use in an automated centrifuge, and 15 milliliters of 30% polyethylene glycol (MW1000) were placed in the second chamber. The container was then subjected to a soft spin of about 580G for three minutes. The platelet-rich plasma thus obtained (23-25 ml) was then decanted to the second chamber and mixed with the PEG. The container was then subjected to hard centrifugation and the supernatant was decanted back to the first chamber. The result was a fibrinogen pellet representing a fibrinogen yield of approximately 70%, a four-to-ten fold increase in TGF-B-1 and a thirty-fold increase in PDGF-AB.

### EXAMPLE 2

Fifty milliliters of whole blood were placed in the first chamber of a container for use in an automated centrifuge, and 7 ml of saturated ammonium sulfate was placed in the second chamber. The container was then subjected to a soft spin of about 580G for three minutes and 23-25 milliliters of platelet-rich plasma were decanted to the second chamber. After mixing with the platelet-rich plasma with the ammonium sulfate, the container was subjected to a hard spin to obtain a fibrinogen pellet, and the supematant decanted to the first chamber. The fibrinogen yield of the pellet was about 72% a four-to-ten fold increase in TGF-B-1 and a thirty-fold increase in PDGF-AB.

Modifications within the scope of the appended claims will be apparent to those of skill in the art.

I claim:

1. A process for isolating growth factor enriched fibrinogen concentrate comprising the steps of:
obtaining platelet rich plasma,
adding a fibrinogen-precipitating agent to said platelet rich plasma; and
recovering growth factor enriched fibrinogen concentrate from said platelet rich plasma.

2. A process according to claim 1 wherein said precipitating agent is polyethylene glycol.

3. A process according to claim 1 wherein said precipitating agent is ammonium sulfate.

4. A process according to claim 1 wherein said step of obtaining platelet rich plasma comprises the step of subjecting plasma to centrifugation.

5. A process according to claim 4 wherein said step of centrifugation comprises the step of subjecting said plasma to a force of about 580G for about three minutes.

6. A process according to claim 1 wherein said platelet rich plasma comprises plasma having 50K to 450K platelets/mm³.

7. A process according to claim 1 wherein said step of recovering fibrinogen comprises the step of subjecting said platelet rich plasma and said precipitating agent to centrifugation.

8. A process according to claim 1 wherein the step of obtaining platelet rich plasma comprises the step of subjecting about 50 ml of anticoagulat d whole blood to centrifugation and decanting 23-25 ml of said platelet rich plasma. and said step of adding a precipitating agent comprises adding about 15 ml of 30% polyethylene glycol (MW1000) to said platelet rich plasma.

9. A process according to claim 1 wherein the step of obtaining platelet rich plasma comprises the step of subjecting about 50 ml of anticoagulated whole blood to centrifugation and decanting 23-25 ml of said platelet rich plasma. and said step of adding a precipitating agent comprises adding about 7 ml of saturated ammonium sulfate.

10. A process according to claim 1 wherein said step of recovering fibrinogen further comprises the step of adding a buffer to said fibrinogen.

11. A product made by the. process of any one of claims 1-10.
